(19) United States
(12) Patent Application Publication
(10) Pub. No.: US 2020/0238048 A1
(43) Pub. Date: Jul. 30, 2020

(54) EASY TO USE URINARY CATHETER

(71) Applicant: Cure Medical LLC, Newport Beach, CA (US)

(72) Inventor: Timothy A. Palmer, Stillwater, MN (US)

(21) Appl. No.: 16/774,784

(22) Filed: Jan. 28, 2020

Related U.S. Application Data
(60) Provisional application No. 62/798,436, filed on Jan. 29, 2019.

(51) Int. Cl. A61M 25/00; B65D 25/28

(52) U.S. Cl. CPC A61M 25/002; A61M 2210/1089; A61M 25/0017; B65D 25/28

(57) ABSTRACT

A number of easy-to-manipulate sterile intermittent urinary catheters have loop-shaped handles attached to proximal funnels, or a sliding sleeve with a loop-shaped handle, or both. In one embodiment, the intermittent urinary catheter may be stored in an easy-to-tear sterile package with finger holes to facilitate grasping and tearing. Sliding sleeves with one-way locking features may also have easy-to-grasp handles.

### RELATED APPLICATIONS

The present application claims priority under 35 USC § 119 to U.S. Provisional Ser. No. 62/798,436, filed Jan. 29, 2019, the contents of which are expressly incorporated herein by reference.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### FIELD

The present invention relates to an intermittent catheter and, more particularly, to improvements in the packaging and design of intermittent catheters to render them easier to use for those with limited dexterity.

### BACKGROUND

The urinary system contains two kidneys, two ureters, the bladder and the urethra. The kidneys filter the blood and produce urine. The urine travels from the kidneys down the ureters and into the bladder, where itis stored until emptied during urination. The urethra is the tube that empties the urine out of the body. When the bladder is full, the brain sends a signal down the spinal cord to the bladder, causing it to empty. Unfortunately, spinal cord injuries such as spina bifida and certain other similar conditions interfere with these signals.

There are an estimated 12,000 spinal cord injuries every year in the United States. More than a quarter of a million Americans are currently living with spinal cord injuries. The largest proportion of spinal cord injuries (36.5%) occurs during car accidents; more than a quarter are the result of falls; and the rest are due to acts of violence (primarily gunshot wounds), sporting accidents, and other less common causes. The average age at injury has risen and is now 42.6 years. 80 percent of spinal cord injury patients are men.

Most spinal cord injuries affect bladder and bowel functions because the nerves that control the involved organs originate in the segments near the lower end of the spinal cord and lose normal brain input. Although the kidneys continue to produce urine, bladder control may be lost and the risk of bladder and urinary tract infections increases.

When people are unable to empty their bladder on their own, they are at risk for urinary tract infections, as well as incontinence or involuntary loss of urine. When urine stays in the bladder and is not emptied, bacteria can grow, causing infections which can lead to illness. Research has shown that intermittent self-catheterization helps reduce urinary tract infections, control urinary leakage (incontinence) and prevent urinary tract damage.

In our highly mobile culture, the ability to have the freedom to leave home for the day or longer is an important part of life. To accommodate this need, single use intermittent catheters have been developed to allow patients to perform self-catheterization. The catheter has one or more small drainage holes on a distal end which can be passed into the bladder, after which point urine flows through the catheter and may be discharged through a proximal outlet funnel. These catheters and their drainage tubes typically have a considerable length and are packaged in an elongated condition. It can require a considerable amount of space to store and transport enough catheters to accommodate a day long outing. Moreover, each catheter must be stored in a sterile condition and administered with care to avoid inadvertent infection, and the process of handling the catheter is often compromised by limited dexterity of the user.

Consequently, there is a need for an easier to use sterile intermittent catheter.

### SUMMARY OF THE INVENTION

The present application provides a number of easy-to-manipulate sterile intermittent urinary catheters. The catheters may have loop-shaped handles attached to proximal funnels, or a sliding sleeve with a loop-shaped handle, or both. In one embodiment, the intermittent urinary catheter may be stored in an easy-to-tear sterile package with finger holes to facilitate grasping and tearing. Sliding sleeves with one-way locking features may also have easy-to-grasp handles.

### BRIEF DESCRIPTION OF THE DRAWINGS

FIG. 1A is a schematic illustration of a package containing a sterile intermittent urinary catheter, and FIG. 1B shows a method for easily opening the package;

FIGS. 1C, 1D and 1E are elevational views of the top edge of two packages similar to that shown in FIGS. 1A and 1B, with alternative finger holes to facilitate opening of the package;

FIG. 2 is a front view of an exemplary funnel found on a proximal end of a sterile intermittent urinary catheter having a grasping ring or handle;

FIG. 3 is a front view of another urinary catheter funnel having two grasping rings or handles on either side thereof;

FIG. 4A is a front view of a further urinary catheter funnel having two handles on either side thereof, and FIG. 4B is a side view of the funnel;

FIG. 5A is a front view of a further urinary catheter funnel having a large single handle on a top thereof, and FIG. 5B is a side view of the funnel;

FIG. 6A is a front view of a further urinary catheter funnel having a smaller single handle on a top thereof, and FIG. 6B is a side view of the funnel;

FIG. 7 is a perspective view of an intermittent urinary catheter having a sliding sleeve with a grasping ring or handle on one side thereof;

FIG. 8 is a partial longitudinal sectional view of the intermittent urinary catheter of FIG. 7 showing the grasping ring or handle on the sliding sleeve;

FIG. 9 is a side view of a distal end of an intermittent urinary catheter having a sliding sleeve of the prior art thereon illustrating its use;

FIG. 10A is a side view of a distal end of an intermittent urinary catheter having an exemplary sliding sleeve with a handle and a one-way locking feature in a position preventing distal movement of sleeve relative to the catheter, and FIG. 10B is a sectional view of the locking feature engaging the catheter;

FIG. 11A is a side view of a distal end of an intermittent urinary catheter having the exemplary sliding sleeve in a position permitting proximal movement of sleeve relative to the catheter, and FIG. 11B is a sectional view of the locking feature disengaged from the catheter; and

FIG. 12A is a longitudinal sectional view of a urinary catheter and another exemplary sliding sleeve with a one-way locking feature thereon, and FIG. 12B is an end view of one example of locking fingers on the sliding sleeve.

### DETAILED DESCRIPTION OF A PREFERRED EMBODIMENT

The present application provides an easy to use sterile intermittent urinary catheter and packaging therefore. The present application provides a number of features that render usage of urinary catheters much easier, in particular for those with limited dexterity. Often spinal injuries which result in the need for urinary catheters are accompanied by partial paralysis of the limbs, and limited use of fingers. The process for manipulating a urinary catheter is somewhat difficult, especially in light of the need to maintain sterility, which makes conventional catheters extremely hard to use for some. The features disclosed herein may be used alone or in combination, and unless mutually exclusive or limited by the claims below each feature can be combined with any other feature. Furthermore, the exemplary urinary catheter illustrated herein is for use by males, but the advantages of the catheter and packaging apply equally to catheters for females.

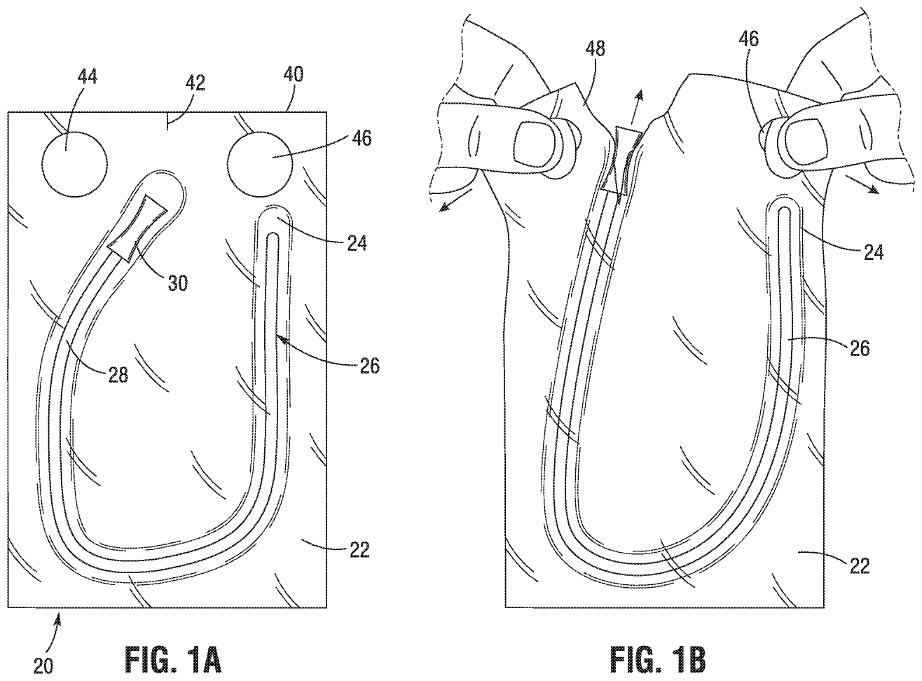

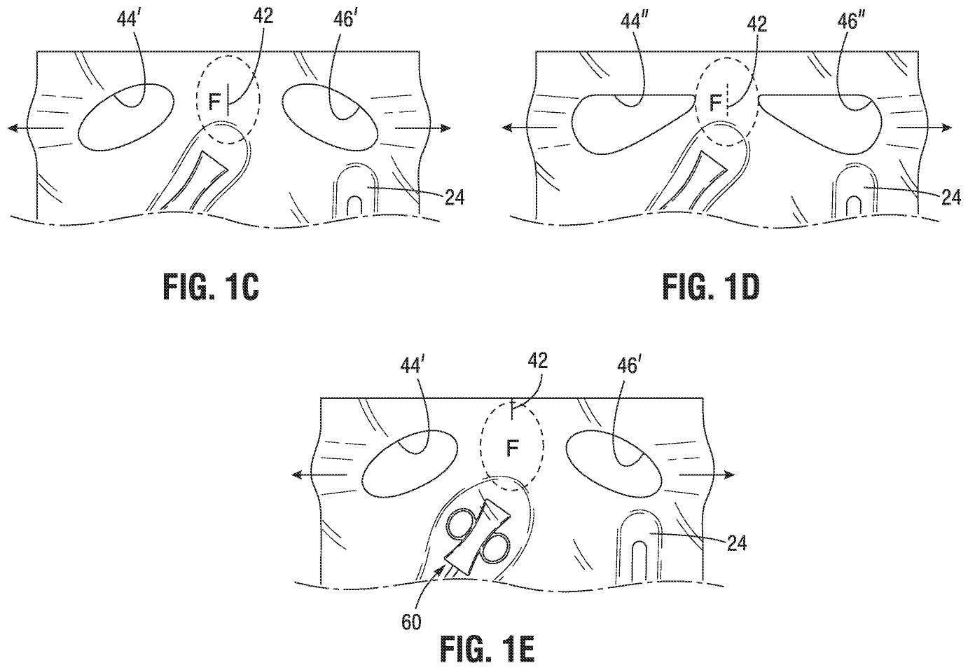

FIG. 1A is a schematic illustration of a packaged sterile intermittent urinary catheter 20 including a flexible package 22 defining an elongated cavity 24 therein. Typically, the package 22 includes two apposed flat panels or layers of thin-film polymer that are secured together such as by heat bonding except at the cavity 24. The cavity 24 is sized and shaped to receive a urinary catheter 26 of between 10-18″ long including an elongated distal tube 28 and a proximal funnel 30.

In the illustrated embodiment, the package 22 is rectangular and has an upper edge 40 that is interrupted by a slit or point of weakness such as the score line 42. A “point of weakness” is understood to be a break in the otherwise homogeneous top edge 40, such as a slit, a perforated score line, a score line that does not extend through the bag material, a notch, etc. A pair of relatively large, round finger holes 44, 46 are formed in the package 22 adjacent each side of the upper edge 40.

FIG. 1B shows a method for easily opening the package 22. A user can hook his or her fingers into the finger holes 44, 46 and pull them apart to create a tear 48 in the upper edge 40 facilitated by the slit 42. The finger holes 44, 46 are positioned to create tension immediately below the slit 42. The slit 42 leads directly to an upper portion of the cavity 24, which is then exposed to the exterior, and the funnel 30 of the urinary catheter 26 can then easily be grasped and pulled from within the package 22.

FIGS. 1C and 1D are elevational views of the top edge of two packages similar to that shown in FIGS. 1A and 1B, with alternative non-circular finger holes to facilitate opening of the package. FIG. 1C shows a package with oval finger holes 44′, 46′ angled so that the upper ends are angled toward each other. This tends to focus the resulting tensile force on the package when pulled apart closer to the upper edge of the bag, as shown by the region F indicated. Desirably, the oval finger holes 44′, 46′ are angled to create the focused tensile region F centered on a point of weakness in the bag, in this case an opening slit 42 located between the top edge of the bag and the cavity 24 in which the catheter is sealed. This is in contrast with simple round holes which when pulled apart generate more diffuse tensile forces between the two holes. Focussing the tensile forces in this way makes it easier to rip the bag open. That is, less force is needed, which may be important for users with compromised dexterity, such as those with severe arthritis. Moving the initiation slit down to the focal point of the opening force as opposed to the top edge also helps, as once the tear is initiated it propagates along its axis in both directions into the catheter cavity 24 and out to the upper edge. Of course, depending on the bag configuration, the point of weakness in the bag may be in the outer edge at a location closest to the inner cavity or between the outer edge and the inner cavity as shown.

Likewise, FIG. 1D shows finger holes 44″, 46″ with shapes resembling two eyes that also focuses the tensile forces toward the slit 42 below the top edge, as shown by the region F indicated. The point of weakness in the bag in this instance is a slit 42 shown in dashed line, indicating a perforated line or line of weakness. The finger holes 44″, 46″ have shapes with generally rounded outer edges which taper narrower toward inner apices pointing at each other, and have a relatively straight top edges parallel with the top edge of the bag. Like the oval finger holes 44′, 46′, the eye-shaped finger holes 44″, 46″ point generally upward and thus toward the slit 42.

FIG. 1E shows the top edge of a package similar to that shown in FIGS. 1A and 1B, with the oval-shaped oval finger holes 44′, 46′ seen in FIG. 1C. Instead of the funnel 30 from the earlier embodiments, this assembly includes a catheter funnel 60 which will be described below with respect to FIG. 3.

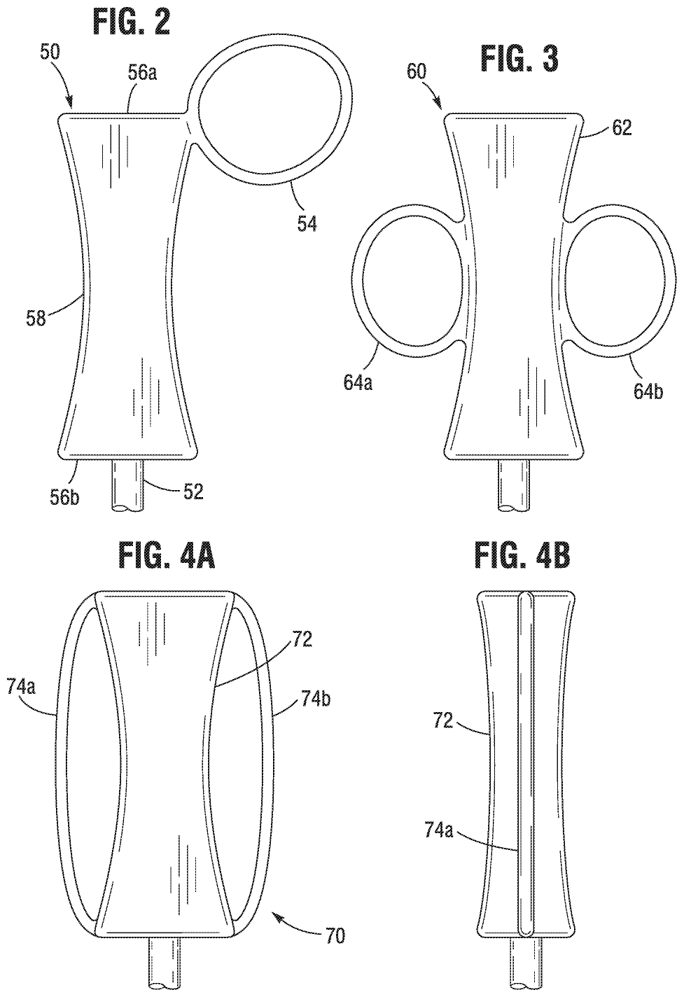

FIG. 2 is a front view of an exemplary funnel 50 (which could be funnel 30 described above) found on a proximal end of a sterile intermittent urinary catheter tube 52, the funnel having a grasping ring or handle 54. The funnel 50 has an open proximal end 56a opposite a distal end 56b into which the catheter tube 52 is fastened, such as with adhesive. A middle portion 58 has concave sides to facilitate grasping by a user. Typically, the funnel 50 is formed of a soft polymer or an elastomeric material for ease of gripping. Some examples of suitable materials include silicone rubber, polyurethanes, thermoplastic elastomers, etc.

Despite the relatively tacky material and concave sides 58 of the funnel 50, many disabled users find it hard to maintain a grip on the funnel during catheter removal or during an extended session of micturition. Consequently, the grasping ring or handle 54 is fastened adjacent the proximal end 56a. In particular, the handle 54 may be made of the same material as the funnel 50 and co-molded therewith (formed as a molded homogeneous material). Alternatively, the handle 54 may be made of the same or different material and adhered to a side edge of the proximal end 56a. In the illustrated embodiment, the handle 54 is a closed circular ring having a sufficient diameter to receive one or two fingers of a user. For example, the handle 54 may be a circular ring of between 1-2 inches in diameter. The handle 54 extends off to the side of the funnel 50 to avoid placing the grasping fingers in the flow path, the funnel sometimes being used as the drain end into a toilet.

FIG. 3 is a front view of another urinary catheter funnel 60 having a body 62 with a concave midportion, and two grasping rings or handles 64a, 64b on either side thereof. Again, the handles 64a, 64b may be circular and sized and constructed the same way as described above for the handle 54. The provision of two handles 64a, 64b allows the user to grasp the funnel 60 on both sides which provides more control for aiming. Again, this funnel 60 is shown in an assembled intermittent urinary catheter package in FIG. 1E.

FIG. 4A is a front view of a further urinary catheter funnel 70 again having a body 72 with a concave midportion, and two handles 74a, 74b on either side thereof. Instead of rings, the two handles 74a, 74b are formed by elongated arched bands that connect proximal and distal ends of the body 72. That is, the two handles 74a, 74b connect the proximal end and distal ends of the body 72, spanning across the concave midportion. Because of the outward arch of the handles 74a, 74b and the concavity of the body 72, relatively large gaps are formed therebetween. FIG. 4B is a side view of the funnel 70 which shows a relatively thin width of one of the handles 74a against a larger width of the body 72. As explained above, the two handles 74a, 74b may be formed of the same material as the body 72 and co-molded therewith, or may be separately attached and made of the same or different material.

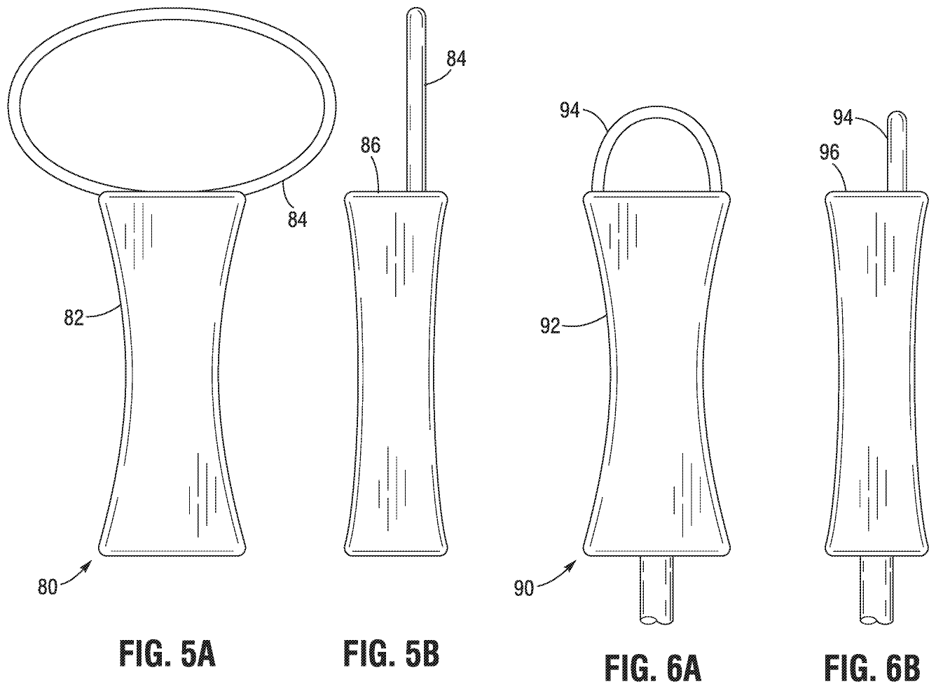

FIG. 5A is a front view of a further urinary catheter funnel 80 with a concave body 82 and having a large single handle 84 on a proximal end thereof, while FIG. 5B is a side view of the funnel. The handle 84 has an oval shape wider than the body 82 and extends from one lateral side of the proximal end. Although the handle 84 is on the proximal end through which urine passes, the handle may be formed of a relatively soft elastomeric material so that the user can bend it out of the way. Furthermore, the user can simply let go of the handle 84 at the time of micturition.

FIG. 6A is a front view of a still further urinary catheter funnel 90 again having a concave body 92 and a smaller single handle 94 extending from a proximal end 96 thereof. FIG. 6B is a side view of the funnel showing the handle 94 attached to one lateral side of the proximal 96. As before, the handle 94 may be co-molded of the same material as the body 92, or the same or different material attached to the body.

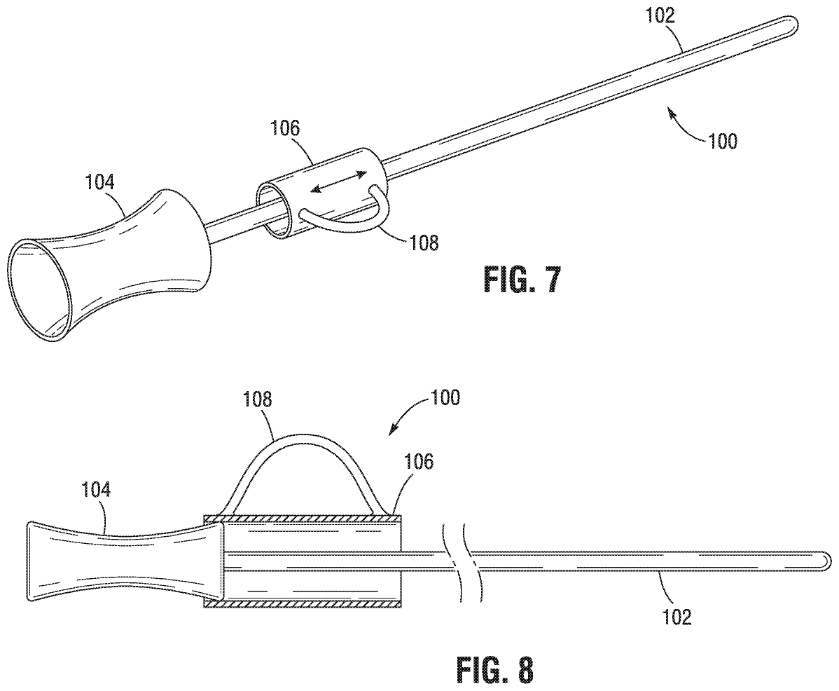

FIG. 7 is a perspective view of an intermittent urinary catheter 100 having an elongated tubular catheter 102 with a funnel 104 attached to a proximal end. As before, the catheter 100 typically has a small drainage hole (not shown) on a distal end which can be passed into the bladder, after which point urine flows through the catheter and may be discharged through the funnel 104. The body of the funnel 104 is again concave for ease of grasping.

A sliding sleeve 106 with a grasping ring or handle 108 on one side thereof is provided over the catheter 102. In the illustrated embodiment, the sliding sleeve 106 is generally tubular and has a diameter which permits it to fit around a distal end of the funnel 104, as seen in FIG. 8, such as during shipment prior to use. The sliding sleeve 106 is formed of a highly flexible material, such as an elastomer, and may be squeezed around the tubular catheter 102. As such, a user may grasp the sliding sleeve 106 and use it to urge the catheter 102 through the urethra into the bladder. This operation is done by squeezing the sleeve 106 and advancing the catheter 102, then releasing the sleeve and retracting it in order to advance another section of the catheter. This assembly helps prevent the user from contacting the typically lubricated outer surface of the catheter 102, which avoids mess and potential contamination.

The handle 108 on the sleeve 106 permits a user to pass a finger or two through the loop provided to help in manipulating the sleeve. The opening formed by the handle 108 may be between 1-2 inches across, for example. Moreover, some users have extreme trouble using their fingers to manipulate the sleeve 106, in which case one or two fingers can be passed through the loop of the handle 108 to enable longitudinal movement of the sleeve, and the other hand can be used to brace the opposite side of the sleeve for squeezing the sleeve around the catheter. It will be understood that any of the handles disclosed herein can be substituted for the single semi-circular handle 108.

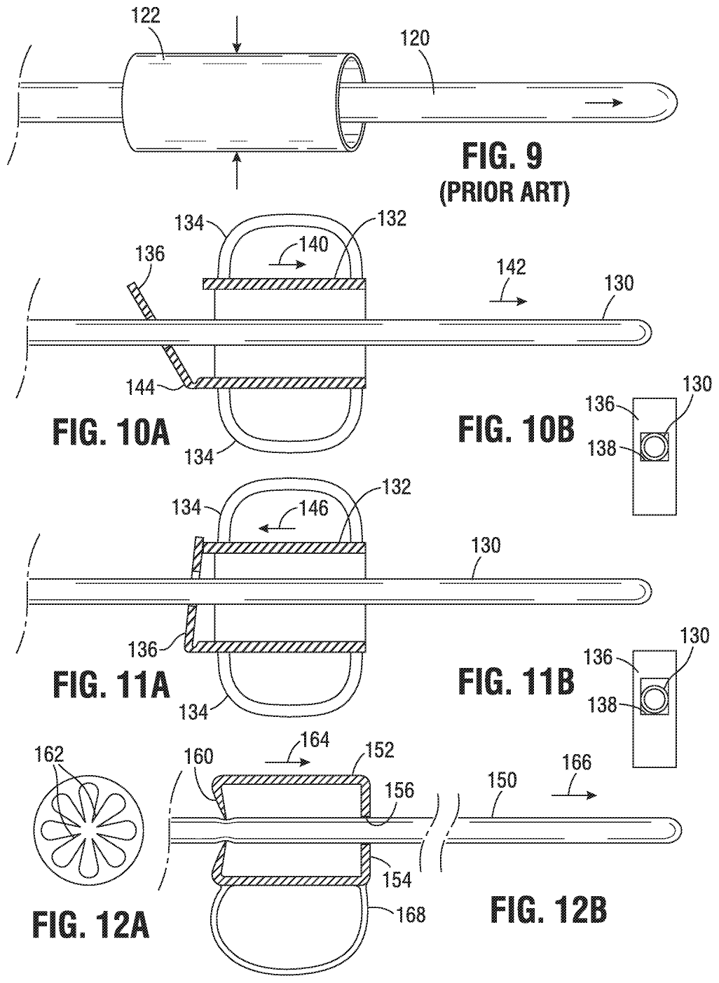

FIG. 9 is a side view of a distal end of an intermittent urinary catheter 120 having a simple tubular sliding sleeve 122 of the prior art thereon and illustrating its use. Namely, the user squeezes the sleeve 122 as indicated by the two arrows and then advances the catheter 120.

FIG. 10A is a side view of a distal end of an intermittent urinary catheter 130 having an exemplary sliding sleeve 132 with a pair of handles 134 extending in opposite lateral directions, and a one-way locking tab 136 pivotally attached to a proximal end thereof. As seen in the end view of FIG. 10B , the locking tab 136 has a through hole 138 formed therein which receives the tubular catheter 130. In FIGS. 10A and 10B, the locking tab 136 is in a misaligned or angled position preventing distal movement of sleeve relative to the catheter. More particularly, as the user displaces the sliding sleeve 132 in a distal direction, as indicated by arrow 140, frictional forces between the catheter and the through hole 138 tend to pivot the locking tab 136 away from the body of the sleeve 132 and from a perpendicular to the angled orientation shown. Eventually, the top and bottom edges of the through hole 138 contact and bind on the catheter 130, which distally displaces the catheter 130 as indicated by arrow 142.

The locking tab 136 is preferably connected to the body of the sliding sleeve 132 via a living hinge 144, and the two may be molded as a single piece. In one embodiment, the through hole 138 is generally rectangular having a height which is slightly greater than the diameter of the catheter 130 such that the through hole 138 binds onto the catheter after pivoting approximately 45°.

FIG. 11A shows the intermittent urinary catheter 130 and exemplary sliding sleeve 132 in a position permitting proximal movement of sleeve relative to the catheter. As the sleeve 132 is displaced in a proximal direction, as indicated by arrow 146, frictional forces between the catheter 130 and the through hole 138 pivot the locking tab 136 back to a generally perpendicular orientation. Because the through hole 138 is larger than the catheter 130, as seen in FIG. 11B, the locking tab 136 no longer binds, and the sleeve 132 can be displaced proximally relative to the catheter. The user can use repeated back and forth movements of the sliding sleeve 132 to incrementally displace the catheter 130 distally into the urethra. Moreover, the pair of handles 134 enable users with less dexterity to easily manipulate the sliding sleeve 132. The pair of handles 134 may be constructed in the same manner as others described herein.

Finally, FIG. 12A is a longitudinal sectional view of a urinary catheter 150 and another exemplary sliding sleeve 152 with a one-way locking feature thereon, and FIG. 12B is an end view of one example of locking fingers on the sliding sleeve. In particular, the sliding sleeve 152 has a generally hollow tubular configuration with a distal face 154 defining a through hole 156 which is greater than the diameter of the catheter 150. A proximal face 160 includes a circular array of locking fingers 162 that extend inwardly toward the catheter and are slightly angled in a proximal direction. The aggregate of the locking fingers 162 defines a through hole that is slightly smaller than the diameter of the catheter 150 when the figures are directly perpendicular to the axis of the catheter.

As the sliding sleeve 152 is displaced in a distal direction, as indicated by arrow 164, friction between the catheter 150 and the locking fingers 162 tends to pivot the fingers to a more perpendicular orientation, which causes them to bind with the catheter. Ultimately, the catheter 150 is urged in a distal direction as indicated by arrow 166. Conversely, opposite proximal movement of the sliding sleeve 152 over the catheter 150 causes the fingers to flex back to their angled orientations, which permits relative movement between the sleeve and catheter. The user can use repeated back and forth movements of the sliding sleeve 152 to displace the catheter 150 distally into the urethra. As before, providing a ring or handle 168 on the sliding sleeve 152 greatly increases the ability of people with reduced dexterity to manipulate the sliding sleeve.

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments.

1. An intermittent urinary catheter package, comprising:
a) a package formed by apposed panels of flexible material between which is defined an inner cavity, the package having an outer edge with a point of weakness in the outer edge at a location closest to the inner cavity or between the outer edge and the inner cavity, the package further having finger holes located on opposite sides of the point of weakness, whereby when a user pulls apart the finger holes tension is created in the package in a region adjacent the point of weakness so as to tear the package through to the inner cavity; and
b) a catheter positioned within the inner cavity, the package being sealed so that the inner cavity remains sterile prior to tearing the package from the point of weakness, the catheter having an elongated flexible tube with at least one open eyelet on a distal end and a funnel attached to a proximal end, wherein distal and proximal define a longitudinal direction.

2. The package of claim 1, wherein the funnel has at least one a loop-shaped handle attached to a central body and sized to receive one or two fingers of a user, the central body having an open proximal end opposite a distal end to which the catheter tube is fastened.

3. The package of claim 2, wherein there are two of the loop-shaped handles extending in opposite lateral directions from the central body.

4. The package of claim 3, wherein the loop-shaped handles and central body are formed of a molded homogeneous soft polymer or elastomeric material.

5. The package of claim 3, wherein the central body has a middle portion with concave sides to facilitate grasping by a user, and the loop-shaped handles extend outward from midpoints of the concave sides.

6. The package of claim 3, wherein the central body has a middle portion with concave sides to facilitate grasping by a user, and the loop-shaped handles connect the proximal end and distal ends spanning across the concave sides.

7. The package of claim 2, wherein there is one loop-shaped handle projecting from the distal end of the central body.

8. The package of claim 7, wherein the loop-shaped handle projects laterally from the distal end of the central body.

9. The package of claim 7, wherein the loop-shaped handle projects longitudinally from the distal end of the central body.

10. The package of claim 9, wherein the loop-shaped handle has an oval shape laterally wider than the body.

11. The package of claim 1, wherein the finger holes are non-circular.

12. The package of claim 11, wherein the finger holes are oval-shaped.

13. The package of claim 1, further including a sliding sleeve disposed around the catheter tube, the sliding sleeve being substantially tubular and sized to freely slide along the catheter tube, the sliding sleeve further including a loop-shaped handle extending laterally therefrom sized to receive one or two fingers of a user.

14. An intermittent urinary catheter package, comprising:
a) a package formed by apposed panels of flexible material between which is defined an inner cavity, the package having an outer edge adjacent the inner cavity configured to permit a user to tear the package through to the inner cavity;
b) a catheter positioned within the inner cavity, the package being sealed so that the inner cavity remains sterile prior to tearing the package from the point of weakness, the catheter having an elongated flexible tube with at least one open eyelet on a distal end and a funnel attached to a proximal end, wherein distal and proximal define a longitudinal direction; and
c) a sliding sleeve disposed around the catheter tube, the sliding sleeve being substantially tubular and sized to freely slide along the catheter tube, the sliding sleeve further including a loop-shaped handle extending laterally therefrom sized to receive one or two fingers of a user.

15. The package of claim 14, wherein the sliding sleeve has two of the loop-shaped handles extending laterally therefrom in opposite directions.

16. The package of claim 14, wherein the sliding sleeve has incorporated therein a one-way locking device which permits relative proximal movement over the catheter tube but inhibits relative distal movement over the catheter tube.

17. The package of claim 16, wherein the one-way locking device is a flap of material hinged to a proximal end of the sliding sleeve and having an aperture through which the catheter tube passes.

18. The package of claim 16, wherein the one-way locking device is a circular array of locking fingers that extend inwardly toward the catheter and are slightly angled in a proximal direction and define an aperture through which the catheter tube passes.

19. The package of claim 14, wherein the sliding sleeve has a diameter which permits it to fit around a distal end of the funnel.

20. The package of claim 14, wherein the package has an outer edge with a point of weakness in the outer edge at a location closest to the inner cavity or between the outer edge and the inner cavity, the package further having non-circular finger holes located on opposite sides of the point of weakness, whereby when a user pulls apart the finger holes tension is created in the package in a region adjacent the point of weakness so as to tear the package through to the inner cavity.
